(19) United States
(12) Patent Application Publication
(10) Pub. No.: US 2020/0249157 A1
(43) Pub. Date: Aug. 6, 2020

(54) System and Method for Secondary Amplification of Refractive Detection Signals

(71) Applicant: UCHICAGO ARGONNE, LLC, Chicago, IL (US)

(72) Inventors: Benjamin Diroll, Chicago, IL (US); Xufeng Zhang, Westmont, IL (US); Supratik Guha, Chicago, IL (US)

(21) Appl. No.: 16/265,638

(22) Filed: Feb. 1, 2019

(51) Int. Cl. G01N 21/43; G01N 33/543

(52) U.S. Cl. CPC G01N 21/431; G01N 2021/432; G01N 33/54373

(57) ABSTRACT

Systems and methods for performing refractometry include a primary waveguide, that has an injection end optically coupled to a laser and an output end optically coupled to a detector. A secondary waveguide has an interior, closed-loop optical path exhibiting total internal reflection, and an exterior surface having bound thereto a plurality of instances of a first binding entity type for an analyte. A portion of the exterior surface of the secondary waveguide is adjacent to the exterior surface of the primary waveguide, and a solution comprising a carrier fluid and an amplification complex includes a secondary particle bound to a second binding entity type for the analyte. The second binding entity type selected to bind to the analyte. The secondary particle is selected to have an index of refraction different from both the carrier fluid and the analyte.

### STATEMENT REGARDING FEDERALLY SPONSORED RESEARCH OR DEVELOPMENT

This invention was made with government support under Contract No. DE-AC02-06CH11357, awarded by the U.S. Department of Energy to UChicago Argonne, LLC, operator of Argonne National Laboratory. The government has certain rights in the invention.

### FIELD OF THE DISCLOSURE

The present disclosure is directed to refractometry and, in particular, to the use of refractometry to detect organic or biological analytes. More particularly, the present disclosure relates to methods and systems for providing secondary amplification of a refractive signal of a detected analyte in optical ring resonators and photonic crystals.

### BACKGROUND

Refractometry is an analytical method in which measurements of the refractive index (indicative of the velocity of light within a medium) of a sample is used as the basis for detection of an analyte. Necessarily, detecting the analyte in the sample requires that the analyte exhibit a refractive index sufficiently different from that of the sample that the detection mechanism can distinguish between the refractive index of the solution and that of the sample. Resonator structures used in refractometry include optical ring resonators and photonic crystals, among others.

In optical ring resonators, the analyte is detected by binding the analyte to a microresonator ring disposed adjacent to a linear waveguide. A laser coupled to an end of the linear waveguide injects light at a specific wavelength into the waveguide, which maintains the light within the linear waveguide due to total internal reflection exhibited thereby. As will be understood, the laser injects the light into the linear waveguide such that the light striking the boundary of the linear waveguide is reflected by, not refracted through, the boundary, because the light falls incident on the boundary at an angle larger than the critical angle and the refractive index on the other side of the boundary is lower than on the side upon which the light is incident. The microresonator ring, also a waveguide, is similarly designed such that it also exhibits total internal reflection for light of the given wavelength.

The microresonator ring is configured to optically couple to the linear waveguide such that part of the light from the linear waveguide couples into the microresonator ring. Any particle or analyte that adsorbs to the external surface of the microresonator ring may affect the refractive index, thereby changing the qualities of the light as it travels around the microresonator. The modified light couples back into the linear waveguide and and is output therefrom so the qualities of the output light can be measured.

Complicating detection of analytes by refractometry is that the range of refractive indices observed in most materials is relatively small. For instance, most visibly-transparent liquids exhibit indices of refraction between 1.3 and 1.5 at visible wavelengths. The discrimination of refractive index places a floor on the detection limit in any technique using an refractometry. When refractometry is used for detection of organic or biological analytes in water, or in materials comprising a high concentration of water, the detection limits are particularly restricted, owing to the similar refractive indices of water and microbial organisms particularly, because the microbial organisms generally are mostly water. Displacement of water in the local environment of the sensor yields a tiny fractional change in the refractive index in the local environment.

Previous efforts to maximize the detectable signal have focused on improving the structure of the refractometry equipment, in particular by decreasing the space between the linear waveguide and a microresonator ring disposed adjacent to the linear waveguide to facilitate increased sensitivity. These methods, unfortunately, can only improve sensitivity by a set amount, owing to physical and manufacturing constraints.

### SUMMARY OF THE DISCLOSURE

A set of described embodiments includes a system for performing refractometry. The system includes a laser configured to output a plurality of optical pulses, each optical pulse having a corresponding selected wavelength and input amplitude. A detector is configured to detect and measure an output signal including an output amplitude at each of the selected wavelengths. A primary waveguide, disposed on a substrate, has an injection end optically coupled to the laser and an output end optically coupled to the detector, and an interior surface configured to exhibit total internal reflection for each of the optical pulses and an exterior surface. A secondary waveguide is also disposed on the substrate, the secondary waveguide having an interior, closed-loop optical path exhibiting total internal reflection at the wavelengths of each of the optical pulses, and an exterior surface having bound thereto a plurality of instances of a first binding entity type for an analyte. The first binding entity type is configured or selected to bind to the analyte when exposed to the analyte. A portion of the exterior surface of the secondary waveguide is adjacent to the exterior surface of the primary waveguide, and a solution comprising a carrier fluid and an amplification complex includes a secondary particle bound to a second binding entity type for the analyte, the second binding entity type selected to bind to the analyte when exposed to the analyte. The secondary particle is selected to have an index of refraction different from both the carrier fluid and the analyte.

Another set of embodiments includes a method of detecting an analyte using an optical ring resonator comprising a primary waveguide and a secondary waveguide. The secondary waveguide includes a closed-loop optical path disposed adjacent to the primary waveguide, and the method includes functionalizing an amplification particle by causing a first binding entity type to bind to the amplification particle. The first binding entity type is selected to bind to both the amplification particle and the analyte. The method also includes exposing the functionalized amplification particle to a carrier fluid containing, or suspected or containing, the analyte, such that the analyte, if present, binds to the functionalized amplification particle. The method further includes functionalizing an exterior surface of the secondary waveguide by causing a second binding entity type to bind to the waveguide, the second binding entity type selected to bind to both the waveguide and the analyte. The method includes exposing the functionalized waveguide to the carrier fluid containing, or suspected of containing, the analyte, such that the analyte, if present, binds to the second binding entity type, and performing refractometry, using the primary and secondary waveguides, to detect the amplification particle.

Another set of embodiments includes a method of detecting an analyte using a photonic resonator structure to perform refractometry. The method includes functionalizing an amplification particle by causing a first binding entity type to bind to the amplification particle, the first binding entity type selected to bind to both the amplification particle and the analyte. The method also includes exposing the functionalized amplification particle to a carrier fluid containing, or suspected or containing, the analyte, such that the analyte, if present, binds to the functionalized amplification particle. The method further includes functionalizing an exterior surface of the photonic resonator structure by causing a second binding entity type to bind to the photonic resonator structure, the second binding entity type selected to bind to both the photonic resonator structure and the analyte. Still further, the method includes exposing the functionalized photonic resonator structure to the carrier fluid containing, or suspected of containing, the analyte, such that the analyte, if present, binds to the second binding entity type, and performing refractometry, using the photonic resonator structure, to detect the amplification particle.

### BRIEF DESCRIPTION OF THE DRAWINGS

The following detailed description will be more easily and better understood when considered in conjunction with the following figures, in which like reference numbers are employed to designate like structures. It should be understood that, with the exception of magnified images, the drawings are not to scale, as scaled drawings would not facilitate an understanding of the depicted structures.

FIG. 1A depicts a perspective view of a silicon photonic chip according to the present description;

FIG. 1B is a top plan view of an example sensor chip having a single sensor circuit;

FIG. 10 is an elevation view along the line A-A of FIG. 1B;

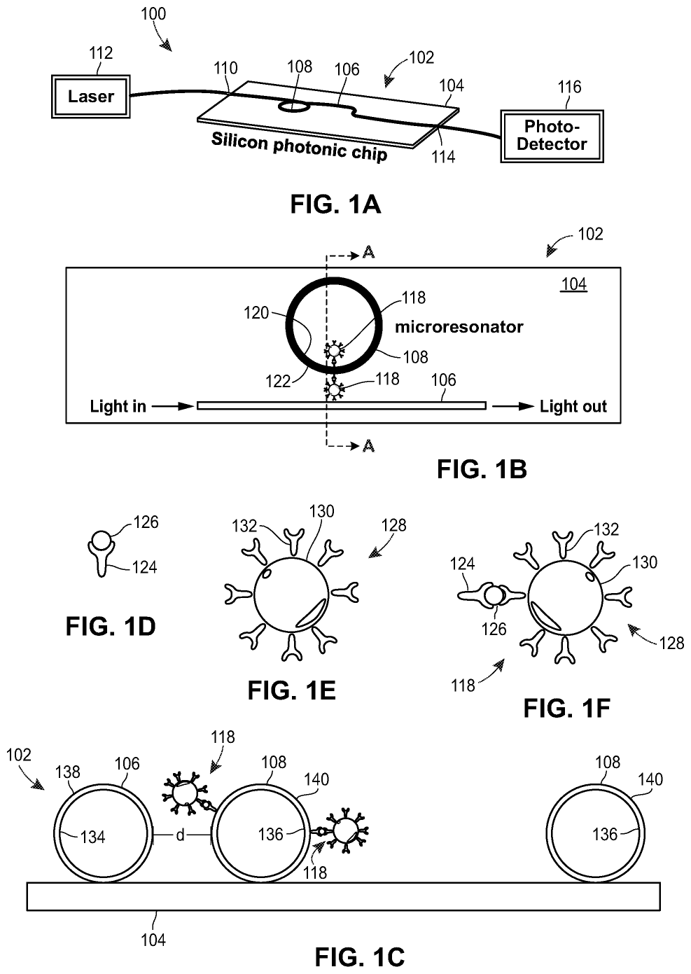

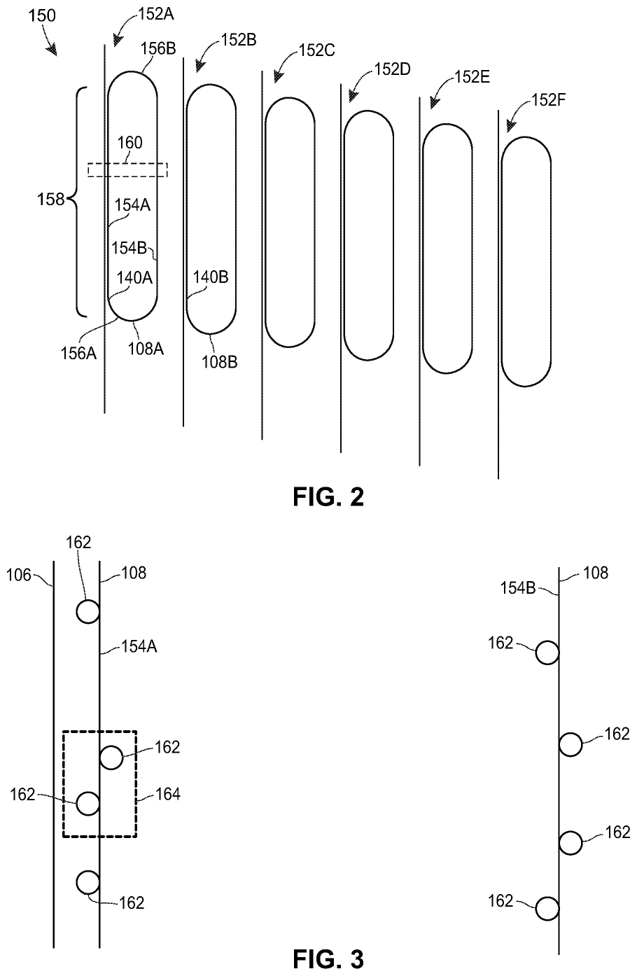

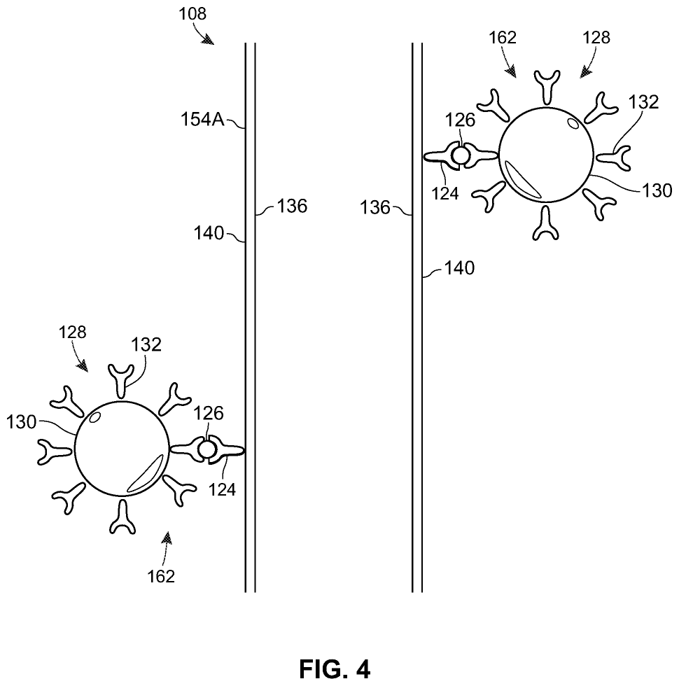

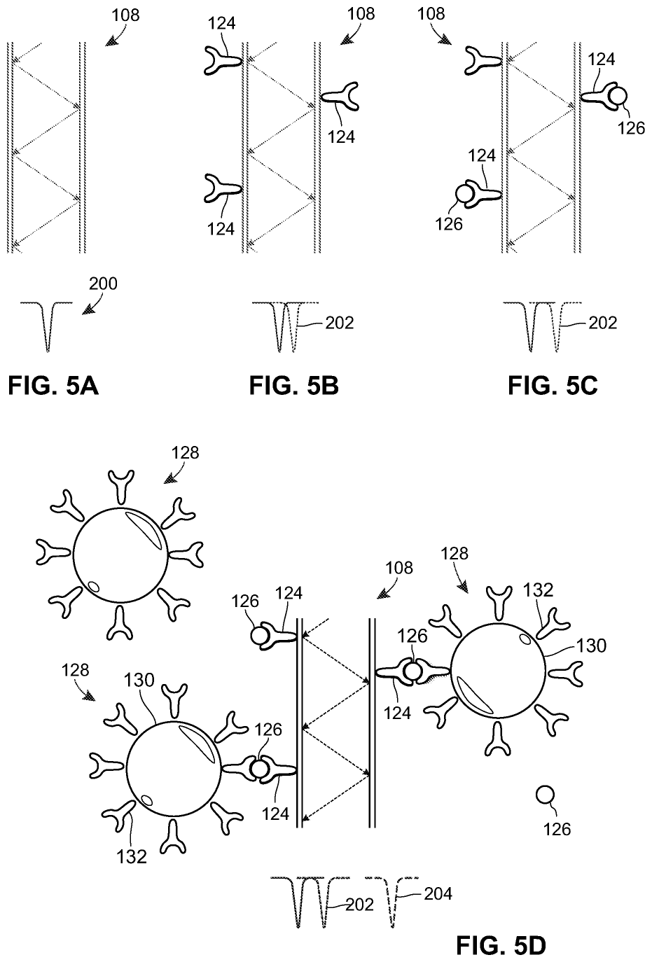

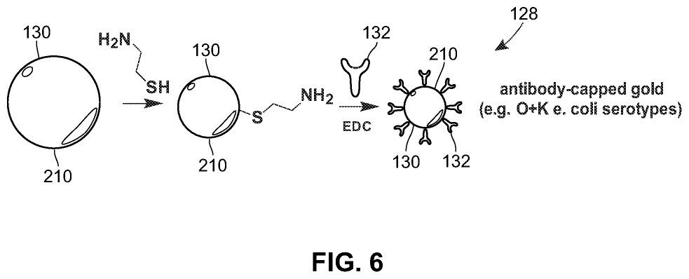

FIG. 1D illustrates an example antibody-antigen complex;

FIG. 1E illustrates an example amplification complex;

FIG. 1F illustrates an example detection complex;

FIG. 2 shows an example arrangement of multiple sensor circuits according to the present disclosure;

FIG. 3 depicts an enlarged (not-to-scale) view of a portion of one of the sensor circuits in FIG. 2;

FIG. 4 depicts a further enlarged (not-to-scale) view of a portion of the enlarged view depicted in FIG. 3;

FIGS. 5A-5D illustrate various example signals associated with the sensor chip in various states of binding with the complexes described herein;

FIG. 6 illustrates an example chemical process for binding antibodies to gold particles;

FIG. 7 is a graph of the real component of indices of refraction for refractometry for water, E. coli, DNA, and gold and silver metals;

FIG. 8A is a graph depicting transmission vs. wavelength for an optical ring resonator in the absence of E. coli or gold;

FIG. 8B is a graph depicting transmission vs. wavelength for an optical ring resonator coated with E. coli;

FIG. 8C is a graph depicting transmission vs. wavelength for an optical ring resonator coated with E coli amplified with gold particles; and

FIGS. 9A-9C depict example methods for detecting an antigen according to the described embodiments.

### DETAILED DESCRIPTION

Methods and systems in accordance with the present description overcome the limitations of detection using only primary binding of the analyte by implementing a secondary binding stage to amplify the change, associated with the analyte, in the index of refraction. Specifically, secondary materials are chemically designed with detection chemistry (e.g., molecular or immunological) for the desired analyte. The secondary materials are specifically chosen to have a large difference in the index of refraction, relative to the carrier medium (e.g., a fluid carrying the analyte) and the analyte, at the relevant detection wavelength. The secondary detection material is then used to selectively bind to the analyte, which has been selectively concentrated in the near environment of the sensor.

By providing an additional and controllably larger change in the index of refraction, the secondary material increases the associated change of refractive index due to the analyte binding to the sensor, and expands the range of analyte concentrations that produce a perceptible change in refractive index. As a result, the use of the secondary material enhances the sensitivity of the device.

FIGS. 1A-1C, which are not to scale, depict a portion of a system 100 for performing refractive detection of analytes using the methods, described herein, for secondary amplification of refractive signals. While described throughout this specification with respect to optical ring resonators, the notional utility of the described embodiments includes other photonic resonators such as, by way of example and not limitation, photonic crystals. With reference to FIG. 1A, which depicts a perspective view of the system 100, the system 100 includes silicon photonic sensor chip 102. The silicon photonic sensor chip (referred to throughout simply as a “sensor chip”) 102 includes a substrate 104 and, disposed on the substrate 104, a primary waveguide 106 and a secondary waveguide 108. The primary waveguide 106 has an injection end 110 optically coupled to a laser 112, and an output end 114 optically coupled to a detector 116. The secondary waveguide 108 is a closed-loop optical path, and is generally disposed such that a portion of the secondary waveguide 108 is adjacent to a corresponding portion of the primary waveguide 106. For the purposes of this disclosure, the respective portions of the primary and secondary waveguides 106, 108 are adjacent when, for the selected wavelength(s) of light, the portions of the primary and secondary waveguides 106, 108 facilitate optical coupling between the portions of the primary and secondary waveguides 106, 108. As will be understood by those of ordinary skill in the art, the primary and secondary waveguides 106, 108 each exhibit total internal reflection at the relevant wavelengths.

The laser 112 is a narrow-band, tunable laser configured to output a series of light pulses, each having a corresponding selected wavelength and amplitude, such that the detector 116 can measure, for each of the selected wavelengths, the received amplitude of the pulse at that wavelength, and therefrom, generate a waveform of transmission vs. wavelength.

FIG. 1B depicts a top plan view of an example sensor chip 102 having a single sensor “circuit” disposed thereon. For the purposes of this description, a sensor circuit comprises a combination of one primary waveguide 106 and one secondary waveguide 108, as depicted in FIGS. 1A-1C. While depicted in FIGS. 1A-1C as having only a single sensor circuit, various embodiments of sensor chips 102 may have multiple sensor circuits disposed thereon, including anywhere from two to 100 or more sensor circuits. Each sensor circuit may be configured to detect the same analyte (e.g., in different samples), or each sensor circuit may be configured detect different analytes (e.g., in the same or different samples).

Detection complexes 118, also depicted in FIG. 1B, are disposed on both an inside exterior surface 120 and an outside exterior surface 122 of the secondary waveguide 108. With reference to FIGS. 1D and 1E, each detection complex 118 includes an antibody 124 (depicted in FIG. 1D) bound to an analyte antigen 126, and an amplification complex 128 (depicted in FIG. 1E) that itself is made up of a secondary particle 130 having one or more second antibodies 132 bound to the secondary particle 130. The second antibodies 132 are selected to bind to the antigen 126 such that the antigen 126 binds to both the antibody 124 and the second antibody 132 to form the detection complex 118 (depicted in FIG. 1F). It should be understood that while described herein in terms of antibodies and antigens, the binding entities corresponding to the antibodies may be any entity that binds to an agent of interest—such as small molecules and/or organic compounds—and the agents of interest corresponding to the antigens may be any compound, molecule, agent, etc. that binds with the selected binding entities.

The first and second antibodies 124, 132 may be the same or different antibodies, in various embodiments. For instance, the first and second antibodies 124, 132 may be the same antibody in cases, for example, where the antigen 126 has two or more binding sites compatible with the same antibody. For example, it will be understood that the family of “avidins,” which include the protein streptavidin, for instance, are tetramer proteins having four binding sites for the same species, biotin. As will be understood, for some antibodies 124, there may be only one complementary binding site on the corresponding antigen 126. In such cases, it will be necessary to use a different antibody, having a different type of complementary binding site on the antigen 126, as the second antibody 132. E. coli, for example, has many protein and carbohydrate types bound to its surface. The first antibody 124 for E. coli may bind to a first one of the protein and carbohydrate types on the surface of the E. coli, while the second antibody 132 may bind to a different one of the protein and carbohydrate types on the surface of the E. coli.

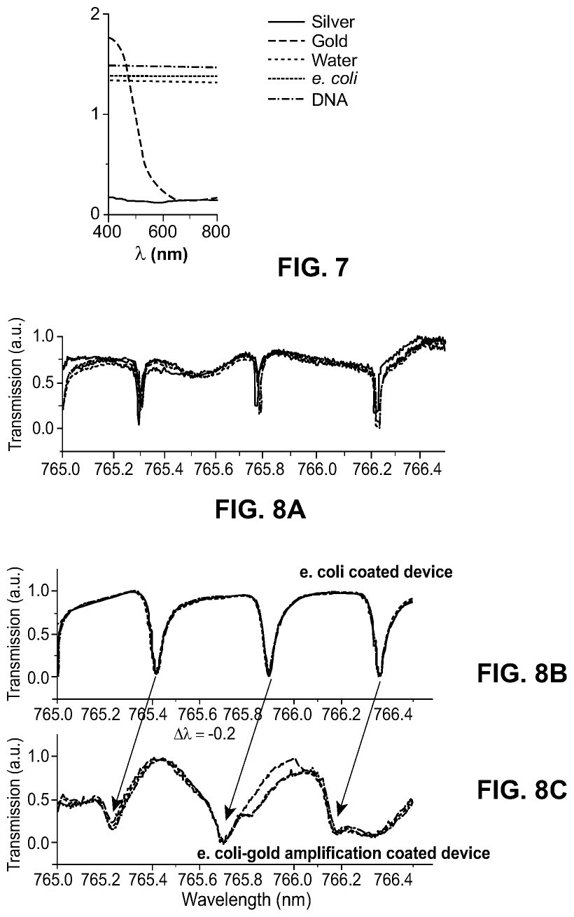

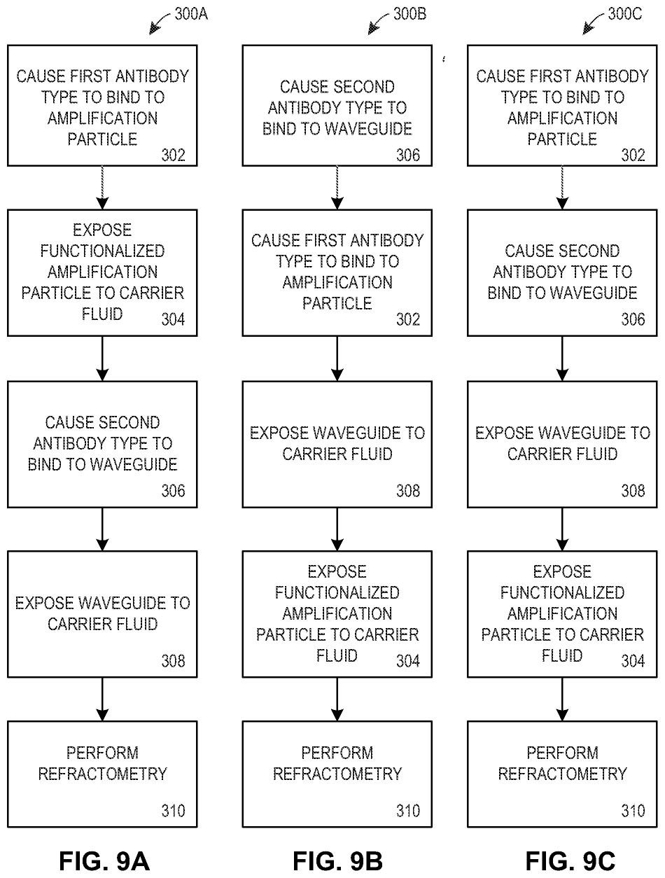

FIG. 10 is an elevation view along the line A-A of FIG. 1B. As depicted in FIG. 10, the primary waveguide 106 and the secondary waveguide 108 are each waveguide structures through which an optical signal can travel by total internal reflection. That is, each of the primary waveguide 106 and the secondary waveguide 108 has an interior surface 134 and 136, respectively, and an exterior surface 138 and 140, respectively. The inside and outside exterior surfaces 120 and 122, respectively, of the secondary waveguide 108, together form the exterior surface 140 of the secondary waveguide 108.

The substrate 104 and the primary and secondary waveguides 106, 108 may be any material or materials suitable for the purpose. By way of example and not limitation, the substrate 104 may be formed from silica-based materials, ceramic materials, silicon materials, or any other material suitable for supporting the waveguides 106, 108 and transparent at the wavelength(s) of interest. Typical waveguide materials may be employed, such as those used in fiber optic applications and, in fact, the waveguides 106, 108 may be fiber-optic in nature, in some embodiments. In other embodiments, the waveguides 106, 108 may be fabricated as part of the substrate 104. Various methods and materials for construction of the substrate 104 and waveguides 106, 108 are known, and the materials employed are only important to the presently described embodiments inasmuch as the materials, specifically of the waveguides 106 and 108, must facilitate attachment thereto of the selected antigens 124. Small molecules and other chemistries may, of course, also enable the attachment to the waveguides 106 and 108 of the selected antigens 124.

While FIGS. 1B and 10 depict only two detection complexes 118 adsorbed to the exterior surface 140 of the secondary waveguide 108, it should be understood that in a typical application, the exterior surface 140 of the secondary waveguide 108 may be coated with any number of antibodies 124. In so doing, each of the antibodies 124 may bind with a corresponding instance of the antigen 126 and, correspondingly, each of the antigens 126 may bind with a corresponding amplification complex 128 so that, collectively, many detection complexes 118 may be present on the exterior surface 140 of the secondary waveguide 108. As a result, the strength of the signal may be correspondingly amplified, resulting in a larger shift of the refractive index and a larger shift in the resonator frequency. The relative intensity of the “transverse electric” (TE) and the “transverse magnetic” (TM) modes—specific resonances—can also be adjusted by the amplified signal. In either case, the signal, be it a wavelength shift or a shift in relative intensity, should increase, indicating a higher concentration or presence of the antigen in question. Of course, it is not necessary that all of the antibodies 124 present on the exterior surface 140 of the secondary waveguide 108 bind to an antigen 126, or that every antigen 126 bound to an antibody 124 also bind to a second antibody 132 of an amplification complex 128.

In embodiments, the distance between the first and second waveguides 106, 108 is minimized in order to maximize the optical coupling between the two. Because the presence of detection complexes 118 between the first and second waveguides 106, 108 maximizes the detectable signal, in some embodiments, the distance, d, between the opposing exterior surfaces 138 and 140 of the waveguides 106 and 108, respectively, is the minimum distance necessary to accommodate detection complexes 118 binding to the secondary waveguide 108 between the waveguides 106, 108. In some embodiments, the distance, d, between the opposing exterior surfaces 138 and 140 of the waveguides 106 and 108, respectively is less than or equal to the median wavelength of the laser pulses output by the laser 112. In some embodiments, the distance, d, between the opposing exterior surfaces 138 and 140 of the waveguides 106 and 108, respectively is less than or equal to the maximum wavelength of the laser pulses output by the laser 112. In some embodiments, the distance, d, between the opposing exterior surfaces 138 and 140 of the waveguides 106 and 108, respectively is less than or equal to the minimum wavelength of the laser pulses output by the laser 112.

FIG. 2 shows an arrangement 150 of several sensor circuits 152A-152F that may be disposed on a substrate (e.g., the substrate 104). The sensor circuits 152A-152F may be configured to detect the same antigens or different antigens. For instance, each of the sensor circuits 152A-152F may be configured to detect different strains of Escherichia coli (E. coli) bacteria by sensitizing each of the sensor circuits 152A-152F with an antibody that selectively binds to a corresponding strain of E. coli. The sensor circuit 152A may have, bound to do the exterior surface 140A of the secondary waveguide 108A, a first antibody that selectively binds to a first strain of E. coli; the sensor circuit 152B may have, bound to the exterior surface 140B of the secondary waveguide 108B, a second antibody that selectively binds to a second strain of E. coli; and so on, so that each of the sensor circuits 152A-152F detects a different strain of E. coli. Of course, different ones of the sensor circuits 152A-152F may also be configured to detect different types of antigens (e.g., the sensor circuit 152A may be configured to detect E. coli, while the sensor circuit 152B may be configured to detect Salmonella, Listeria, or the like).

While in some embodiments, such as that depicted in FIG. 1B, the secondary waveguide 108 may take the form of an annulus, in other embodiments, the secondary waveguide 108 may take the form of a non-annular, closed-loop figure, such as those depicted in FIG. 2. In the sensor circuits 152A-152F depicted in FIG. 2, the secondary waveguide 108 takes the form of a racetrack, having two generally linear sides 154A and 154B connected by opposing curved portions 156A and 156B that, in FIG. 2, have a generally semi-circular shape. Advantageously, the use of the linear sides 154A, 154B increases the length 158 over which the primary waveguide 106 and the secondary waveguide 108 may be in close proximity to one another and, as a result, increases the effects of optical coupling between the primary waveguide 106 and the secondary waveguide 108.

FIG. 3 depicts a (not to scale) enlarged view of a portion 160 of the sensor circuit 152A depicted in FIG. 2. The figure depicts the primary waveguide 106 in close proximity with the linear side 154A of the secondary waveguide 108, while the linear side 154B is further from the secondary waveguide 108. Both the linear side 154A and the linear side 154B have detection complexes 162 coupled to the exterior surface 140 thereof. It will be understood that, while FIG. 3 depicts only a few detection complexes 162, any given region of the sensor circuits in the contemplated embodiment may have a large number (tens, hundreds, thousands, or more, depending on the surface area in question) of antibodies 124 disposed on the exterior surface 140 of the secondary waveguide 108. Additionally, while not depicted in FIG. 3, in embodiments the primary waveguide 106 may also have, bound to its exterior surface 138, antibodies 124, to which antigens 126 may also bind. While the detection complexes 118 bound to the primary waveguide 106 may not contribute to as significantly to the detectable signal as the detection complexes 118 bound to the secondary waveguide 108 (depicted in FIG. 3 as detection complexes 162), it may be impractical to prevent detection complexes 118 from binding to the exterior surface 138 of the primary waveguide 106.

FIG. 4 depicts a further enlarged (not to scale) view of a portion 164 of the portion 160 depicted in FIG. 3. Detection complexes 162 are bound to the exterior surface 140 of the secondary waveguide 108. The binding of detection complexes can be accomplished in several ways. Generally, however, the exterior surface 140 of the secondary waveguide 108 must be treated such that the antibodies 124 bind to the surface 140. For example, the exterior surface 140 may be coated with a silane (aminopropyltriethoxylsilane), which forms a bond to the silicon oxide surface 140 and presents an amine functional group. Then, the amine functional group is reacted with carboxylate functional groups on a protein to form an amide bond. That is, a covalent bond is formed. Other possible routes involve amide bond formation, but reversing the order of chemical functions used (e.g., protein with amine, surface with carboxylate), click chemistry reaction, and non-covalent adsorption, such as electrostatic or van der waals binding. In any event, thereafter, the surface 140 is exposed to the antibody 124 to allow the antibody 124 to bind to it. When the antibody 124 binds to the surface 140, the antibody 124 may be unbound to the antigen 126, or may be already bound to the antigen 126. At the same time, the antigen 126 may be bound already to the amplification complex 128 or may be unbound to the amplification complex 128. In any event, the antibody 124—whether or not bound to other structures—binds to the surface 140 of the secondary waveguide 108. As should be apparent, in embodiments, the surface 138 of the primary waveguide 106 may also be treated such that the antibodies 124 bind thereto and, when exposed to the antibodies 124, the antibodies 124—whether or not bound to other structures—may also bind to the surface 138 of the primary waveguide 106.

As will be understood, the specific region on an antigen that an antibody recognizes and binds to is called the epitope, or antigenic determinant. An epitope is usually 5-8 amino acids long on the surface of the protein. Proteins are three dimensionally folded structures, and an epitope may only be recognized in its form as it exists in solution, or its native form. When an epitope is made up of amino acids that are brought together by the three-dimensional structure, the epitope is conformational, or discontinuous. If the epitope exists on a single polypeptide chain, it is a continuous, or linear epitope. Depending on the epitope an antibody recognizes, it may bind only fragments of a protein, or it may also be able to bind the native protein.

The specific association of an antigen with an antibody is dependent on hydrogen bonds, hydrophobic interactions, electrostatic forces, and Van der Waals forces. These are of a weak, noncovalent nature, yet some of the associations between antigen and antibody can be quite strong. Like antibodies, antigens can be multivalent, either through multiple copies of the same epitope, or through the presence of multiple epitopes that are recognized by multiple antibodies. Interactions involving multivalency can produce more stabilized complexes; however, multivalency can also result in steric difficulties, thus reducing the possibility for binding. All antigen antibody binding is reversible and follows the basic thermodynamic principles of any reversible bimolecular interaction: where KA is the affinity constant, [Ab−Ag] is the molar concentration of the antibody-antigen complex, and [Ab] and [Ag] are the molar concentrations of unoccupied binding sites on the antibody (Ab) or antigen (Ag), respectively.

The strength of interaction between antibody and antigen at single antigenic sites can be described by the affinity of the antibody for the antigen. Within each antigenic site, the variable region of the antibody “arm” interacts through weak noncovalent forces with antigen at numerous sites. The greater the interaction, the stronger the affinity. Avidity is perhaps a more informative measure of the overall stability or strength of the antibody-antigen complex. It is controlled by three major factors: antibody epitope affinity, the valence of both the antigen and antibody, and the structural arrangement of the interacting parts. Ultimately these factors define the specificity of the antibody, that is, the likelihood that the particular antibody is binding to a precise antigen epitope.

As a result of these varying factors, it will also be understood that the first and second antibodies 124 and 132 may be the same or different, depending on whether the antigen 126 has multiple epitopes recognized by the same antibody. Where the antigen 126 has multiple epitopes that can be recognized by the same antibody, the antibodies 124 and 132 may be the same. Alternatively, where the antigen 126 has only a single epitope that is recognized by the antibody 124, the antibody 132 must necessarily be a different antibody that recognizes a different epitope on the antigen 126. Further, as should be understood, the antibody 124 and the antibody 132 will be selected according to the particular antigen 126.

As will be understood, the sensors described herein will exhibit light transmission “valleys” (i.e., decreases in transmission) at specific wavelengths that depend on the index of refraction of the environment. With reference now to FIGS. 5A-5D, the presence of various elements in the vicinity of the sensor circuit will, as discussed above, alter the signal output from output end 114 of the primary waveguide 106 and detected by the detector 116, by changing the characteristics of the “valleys.” The signals may decrease the light transmission across a band of wavelengths, which may result in a more shallow “valley,” may shift the wavelengths over which the light transmission is decreased (moving the “valley” in one direction or the other along the wavelength spectrum), and/or may change the set of wavelengths over which light transmission is decreased, causing the “valley” to become broader or narrower.

Generally, waveguides 106, 108 are exposed to the various elements by exposing the sensor chip 102 to a fluid containing the elements, and generally (though not always) that fluid will have a high water content. FIG. 5A depicts a generic signal 200 that could be detected when no antibodies are bound to the secondary waveguide 108. That signal 200 may shift slightly (indicated by the dotted line 202) in the presence of the antibodies 124 that are not yet bound to antigens 126, as depicted in FIG. 5B. When antigens 126 bind to antibodies 124, the observed signal 202 may shift slightly further (as depicted in FIG. 5C). The signal 202 in FIG. 5C generally represents the signal that would be observed in the absence of the secondary amplification features described herein and, when the antibody 126 exhibits a refractive index close to that of the medium in which it sits (as is the case with a biological cell—e.g., E. coli, Salmonella, Listeria, or the like—in a water-based medium), it may be difficult to discern from the signal that the antigen 126 is present.

FIG. 5D, however, depicts the instance in which the detection complex 118, including the amplification complex 128 comprising the secondary particle 130, is bound to the secondary waveguide 108. The secondary particle 130, being selected to have a refractive index significantly different than that of the medium or the antigen 126 causes the output to shift significantly, presenting a readily detectable, amplified signal, as can be seen in the difference between the signal 202 (without the amplification complex 128) and a signal 204 (with the amplification complex 128).

The described embodiments may be implemented to detect any number of analyte antigens, including virii, bacteria, proteins, and the like, simply by functionalizing the exterior surface 140 of the secondary waveguide 108 to bind to an antibody specifically selected to bind to the analyte of interest. Thus, the described embodiments may be configured to detect antigens such as, by way of example and not limitation, viral antigens associated with virus such as, but not limited to, influenza A, influenza B, herpes, smallpox, measles, mumps, rubella, hepatitis A, hepatitis B, hepatitis C, rabies, Ebola, Hanta fever, chicken pox, polio, HIV, Zika, etc.; or bacterial antigens associated with bacteria such as, but not limited to, E. coli, Helicobacter pylori, Staphylococcus, Streptococcus, Pneumococcus, Salmonella, Listeria, Anthrax, etc. The described embodiments may also be implemented to detect an antigen in any variety of media, including air, water, bodily fluids, such as blood, urine, serum, plasma, saliva, mucus, tears, cerebrospinal fluid, vaginal fluid, semen, sputum, and various buffer solutions and growth media.

In contemplated embodiments, the secondary particle 130 exhibits an index of refraction differing from that of the antigen 126 and from that of the medium in which the antigen 126 is present by an amount that induces a change in output signal (relative to prior art detection of the antigen-antibody complex alone) that increases detectability of the antigen of interest, decreases the absolute quantity of antigen that must be present in order to detect its presence, or decreases the concentration of the antigen necessary to detect its presence. In various embodiments, the secondary particle 130 increases the detectability of the antigen, or decreases the quantity or concentration of the antigen necessary to detect it, by more than 5%, by more than 10%, by more than 15%, by more than 20%, or by more than 50%. In various embodiments, the detectability of the antigen of interest is increased by selecting a secondary particle 130 that has an index of refraction differing from both the antigen 126 and the medium in which the antigen 126 is present by more than 0.1, by more than 0.2, by more than 0.3, by more than 0.4, by more than 0.5, by more than 0.75, or by more than 1.0.

In some aspects, an antibody 132 is conjugated to a secondary particle 130. The conjugation of the secondary particle 130 with an antibody 132 combines the properties of the secondary particle 130 with the specific and selective recognition ability of the antibody 132 to the antigen 126. In some embodiments, the secondary particle 130 may be metallic. Such embodiments may include those in which the secondary particle 130 includes silver, gold, copper, iron, magnesium, sodium, and the like. In other embodiments, the secondary particle 130 may be silicon or a silicate, or may be a glass bead. In some exemplary embodiments, the secondary particle 130 is a gold nanoparticle. In various aspects, the conjugation of the antibody 132 to the secondary particle 130, or vice versa, is carried out by physical, chemical or biological means, including, but not limited to those means known in the art or as described by Jazayeri et al. (Sensing and Bio-Sensing Research 9:17-22, 2016) and Arruebo et al., (J. Nanomaterials, Volume 2009, Article ID439389, 24 pages).

By way of example, for a typical optical ring microresonator structure, which has an optical transmission mode near 700 nm, gold is an excellent material for use as a secondary particle 130 when detecting E. coli in water, because gold at 700 nm exhibits an index of refraction of 0.14, while that of water is 1.33 and that of E. coli is 1.38.

FIG. 6 depicts an example strategy for functionalizing the secondary particle 130 to bind to the antigen 132 via the intermediary second antibody 132 and, in particular, depicts a strategy for functionalizing gold particles 210. Gold particles are synthesized in water by boiling in the presence of a citrate. The particles are subsequently exposed to a bifunctional molecule, such as cysteamine, that enables both adsorption to the particle surface (which occurs via the thiol (sulfur)) and subsequent chemical reaction with the amine functional group. The amine functional group is covalently bound through the formation of an amide bond with a chosen antibody catalyzed by 1-Ethyl-3-(3-dimethylaminopropyl)carbodiimide (EDC). Of course, FIG. 6 depicts a specific strategy for functionalizing a specific secondary particle, and is merely exemplary in nature, as the benefits of the described embodiments are agnostic with respect to how the secondary particle 130 is functionalized.

FIG. 7 is a graph of the real component of the indices of refraction for refractometry for water, E. coli, DNA, and gold and silver metals. As can be seen in the graph, the indices of refraction for water, E. coli, and DNA are all relatively stable over the range of wavelengths from 400-800 nm, and all relatively close to one another. The index of refraction of gold metal decreases precipitously around 500 nm wavelengths and remains significantly lower than water, E. coli, and DNA at higher wavelengths. The index of refraction of silver metal is and remains significantly lower than water, E. coli, and DNA over the 400-800 nm range of wavelengths.

FIGS. 8A to 8C are graphs depicting transmission vs. wavelength curves for an experimental setup for detecting E. coli in water, using gold particles for amplification, in accordance with described embodiments. FIG. 8A shows the transmission vs. wavelength curve for an optical ring resonator in the absence of any E. coli or gold. Distinct, but narrow, dips in the signal can be observed at 765.3 nm, 765.8 nm, and at around 766.25 nm. When the resonator is exposed to E. coli so that antibodies on the surface of the secondary waveguide have bound the antigen to the waveguide, the transmission vs. wavelength curves change, as illustrated in FIG. 8B. While three distinct dips are still observed in FIG. 8C, each is wider than in FIG. 8A, and each is shifted approximately 0.1 nm toward higher wavelengths (765.4 nm, 765.9 nm, and 766.35 nm). However, when the resonator is exposed to E. coli coupled to gold particles for amplification as described herein, the transmission vs. wavelength curves change drastically, as illustrated in FIG. 8C. The dips become drastically wider and shift approximately 0.2 nm toward lower wavelengths.

FIGS. 9A-9C depict methods 300A-300C for detecting an antigen according to the described embodiments. While the methods 300A-300C each have the same steps, the methods are depicted to illustrate that the precise order of the steps may change, as long as certain pre-conditions are met. With reference to FIG. 9A, the method includes causing a first antibody type to bind to the amplification particle (block 302), exposing the functionalized amplification particle to carrier fluid containing, or suspected of containing, the antigen (block 304), causing a second antibody type to bind to the (secondary) waveguide (block 306), exposing the (secondary) waveguide to the carrier fluid (block 308), and performing refractometry (block 310). FIGS. 9B and 9C illustrate that, as long as block 302 occurs before block 304, as long as block 306 occurs before block 308, and as long as all of blocks 302-308 occur before block 310, the precise order in which these steps are performed is immaterial.

In some embodiments the methods 300A-300C may also include a step (not shown) of removing any amplification particles that are not bound to an antigen. By doing so, amplification particles that are not bound to an antigen will not by chance be in the area of the sensor circuit when refractometry is performed. As a result, such amplification particles will not be able to cause false positives by being detected even though not bound to an antigen.

The following list of aspects reflects a variety of the embodiments explicitly contemplated by the present application. Those of ordinary skill in the art will readily appreciate that the aspects below are neither limiting of the embodiments disclosed herein, nor exhaustive of all of the embodiments conceivable from the disclosure above, but are instead meant to be exemplary in nature.

1. A system for performing refractometry, comprising: a laser configured to output a plurality of optical pulses, each optical pulse having a corresponding selected wavelength and input amplitude; a detector configured to detect and measure an output signal, the output signal comprising an output amplitude at each of the selected wavelengths; a primary waveguide, disposed on a substrate, the primary waveguide having an injection end optically coupled to the laser and an output end optically coupled to the detector, the primary waveguide having an interior surface configured to exhibit total internal reflection for each of the optical pulses and an exterior surface; a secondary waveguide disposed on the substrate, the secondary waveguide comprising an interior, closed-loop optical path exhibiting total internal reflection at the wavelengths of each of the optical pulses, and an exterior surface having bound thereto a plurality of instances of a first binding entity type for an analyte, the first binding entity type configured to bind to the analyte when exposed to the analyte, wherein a portion of the exterior surface of the secondary waveguide is adjacent to the exterior surface of the primary waveguide; and a solution comprising a carrier fluid and an amplification complex, the amplification complex comprising a secondary particle bound to a second binding entity type for the analyte, the second binding entity type selected to bind to the analyte when exposed to the analyte, wherein the secondary particle is selected to have an index of refraction different from both the carrier fluid and the analyte.

2. A system according to aspect 1, wherein the analyte is a bacterial antigen.

3. A system according to aspect 2, wherein the bacterial antigen is from Escherichia coli.

4. A system according to aspect 1, wherein the analyte is a viral antigen.

5. A system according to aspect 1, wherein the analyte is a protein.

6. A system according to any one of aspects 1 to 5, wherein the secondary particle comprises a metal.

7. A system according to any one of aspects 1 to 5, wherein the secondary particle comprises silver.

8. A system according to any one of aspects 1 to 5, wherein the secondary particle comprises gold.

9. A system according to any one of aspect 1 to 5, wherein the secondary particle comprises silicon.

10. A system according to any one of aspects 1 to 5, wherein the secondary particle comprises a silicate.

11. A system according to aspect 11, wherein the silicate is glass.

12. A system according to any one of aspects 1 to 11, wherein the first and second binding entity types are different from one another.

13. A system according to any one of aspects 1 to 11, wherein the first and second binding entity types are the same.

14. A system according to any one of aspects 1 to 13, wherein the primary and secondary waveguides each have an interior surface and an exterior surface, and wherein the interior surfaces each exhibit total internal reflection at the selected wavelengths.

15. A system according to aspect 14, wherein the minimum distance between the primary and secondary waveguides is selected to maximize optical coupling between the first and second waveguides.

16. A system according to any one of aspects 1 to 15, wherein the secondary particle is selected to have an index of refraction differing from both the carrier fluid and the analyte by more than 0.1.

17. A system according to any one of aspects 1 to 15, wherein the secondary particle is selected to have an index of refraction differing from both the carrier fluid and the analyte by more than 0.25.

18. A system according to any one of aspects 1 to 15, wherein the secondary particle is selected to have an index of refraction differing from both the carrier fluid and the analyte by more than 0.5.

19. A system according to any one of aspects 1 to 15, wherein the secondary particle is selected to have an index of refraction differing from both the carrier fluid and the analyte by more than 1.0.

20. A system according to any one of aspects 1 to 19, wherein the carrier fluid is water.

21. A system according to any one of aspects 1 to 19, wherein the carrier fluid is a bodily fluid.

22. A system according to aspect 21, wherein the bodily fluid is blood.

23. A system according to aspect 21, wherein the bodily fluid is urine.

24. A system according to any one of aspects 1 to 19, wherein the carrier fluid is a buffer solution.

25. A system according to any one of aspects 1 to 19, wherein the carrier fluid is air.

26. A method of detecting an analyte using an optical ring resonator comprising a primary waveguide and a secondary waveguide, the secondary waveguide comprising a closed-loop optical path disposed adjacent to the primary waveguide, the method comprising: functionalizing an amplification particle by causing a first binding entity type to bind to the amplification particle, the first binding entity type selected to bind to both the amplification particle and the analyte; exposing the functionalized amplification particle to a carrier fluid containing, or suspected or containing, the analyte, such that the analyte, if present, binds to the functionalized amplification particle; functionalizing an exterior surface of the secondary waveguide by causing a second binding entity type to bind to the waveguide, the second binding entity type selected to bind to both the waveguide and the analyte; exposing the functionalized waveguide to the carrier fluid containing, or suspected of containing, the analyte, such that the analyte, if present, binds to the second binding entity type; and performing refractometry, using the primary and secondary waveguides, to detect the amplification particle.

27. A method according to aspect 26, wherein performing refractometry to detect the amplification particle comprises: injecting a series of laser optical pulses into a first end of the primary waveguide, each optical pulse having a corresponding selected wavelength and amplitude; measuring at a detector optically coupled to a second end of the primary waveguide, the output amplitude of each of the pulses to determine a transmission value at each of the selected wavelengths; and comparing the transmission values at each of the selected wavelengths to corresponding transmission values obtained prior to exposing the functionalized waveguide to the carrier fluid.

28. A method according to either aspect 26 or aspect 27, wherein the analyte is a bacterial antigen.

29. A method according to aspect 28, wherein the bacterial antigen is from Escherichia coli.

30. A method according to either aspect 26 or aspect 27, wherein the analyte is a viral antigen.

31. A method according to either aspect 26 or aspect 27, wherein the analyte is protein.

32. A method according to any one of aspects 26 to 31, wherein the amplification particle comprises a metal.

33. A method according to any one of aspects 26 to 31, wherein the amplification particle comprises silver.

34. A method according to any one of aspects 26 to 31, wherein the amplification particle comprises gold.

35. A method according to any one of aspects 26 to 31, wherein the amplification particle comprises silicon.

36. A method according to any one of aspects 26 to 31, wherein the amplification particle comprises a silicate.

37. A method according to aspect 36, wherein the silicate is glass.

38. A method according to any one of aspects 26 to 37, wherein the first and second binding entity types are different from one another.

39. A method according to any one of aspects 26 to 37, wherein the first and second binding entity types are the same.

40. A method according to any one of aspects 26 to 39, wherein the primary and secondary waveguides each have an interior surface and an exterior surface, and where in the interior surfaces each exhibit total internal reflection at the selected wavelengths.

41. A method according to aspect 40, wherein the minimum distance between the primary and secondary waveguides is selected to maximize optical coupling between the first and second waveguides.

42. A method according to any one of aspects 26 to 41, further comprising electing the amplification particle to have an index of refraction differing from both the carrier fluid and analyte by more than 0.1.

43. A method according to any one of aspects 26 to 41, further comprising electing the amplification particle to have an index of refraction differing from both the carrier fluid and analyte by more than 0.25.

44. A method according to any one of aspects 26 to 41, further comprising electing the amplification particle to have an index of refraction differing from both the carrier fluid and analyte by more than 0.5.

45. A method according to any one of aspects 26 to 41, further comprising electing the amplification particle to have an index of refraction differing from both the carrier fluid and analyte by more than 1.0.

46. A method according to any one of aspects 26 to 45, wherein the carrier fluid is water.

47. A method according to any one of aspects 26 to 45, wherein the carrier fluid is a bodily fluid.

48. A method according to aspect 47, wherein the bodily fluid is blood.

49. A method according to aspect 47, wherein the bodily fluid is urine.

50. A method according to any one of aspects 26 to 45, wherein the carrier fluid is a buffer solution.

51. A method according to any one of aspects 26 to 45, wherein the carrier fluid is air.

52. A system according to any one of aspects 1 to 25, wherein the analyte is an antigen.

53. A system according to any one of aspects 1 to 25 or 52, wherein the first binding entity type is an antibody.

54. A system according to any one of aspects 1 to 25, 52, or 53, wherein the second binding entity type is an antibody.

55. A method according to any one of aspects 26 to 51, wherein the analyte is an antigen.

56. A method according to any one of aspects 26 to 51 or 55, wherein the first binding entity type is an antibody.

57. A method according to any one of aspects 26 to 51, 55, or 56, wherein the second binding entity type is an antibody.

58. A method of detecting an analyte using a photonic resonator structure to perform refractometry, the method comprising: functionalizing an amplification particle by causing a first binding entity type to bind to the amplification particle, the first binding entity type selected to bind to both the amplification particle and the analyte; exposing the functionalized amplification particle to a carrier fluid containing, or suspected or containing, the analyte, such that the analyte, if present, binds to the functionalized amplification particle; functionalizing an exterior surface of the photonic resonator structure by causing a second binding entity type to bind to the photonic resonator structure, the second binding entity type selected to bind to both the photonic resonator structure and the analyte; exposing the functionalized photonic resonator structure to the carrier fluid containing, or suspected of containing, the analyte, such that the analyte, if present, binds to the second binding entity type; and performing refractometry, using the photonic resonator structure, to detect the amplification particle.

59. A method according to aspect 58, wherein the analyte is a bacterial antigen.

60. A method according to aspect 58, wherein the bacterial antigen is from Escherichia coli.

61. A method according to aspect 58, wherein the analyte is a viral antigen.

62. A method according to aspect 58, wherein the analyte is protein.

63. A method according to any one of aspects 58 to 62, wherein the amplification particle comprises a metal.

64. A method according to any one of aspects 58 to 63, wherein the amplification particle comprises silver.

65. A method according to any one of aspects 58 to 64, wherein the amplification particle comprises gold.

66. A method according to any one of aspects 58 to 65, wherein the amplification particle comprises silicon.

67. A method according to any one of aspects 58 to 66, wherein the amplification particle comprises a silicate.

68. A method according to aspect 67, wherein the silicate is glass.

69. A method according to any one of aspects 58 to 68, wherein the first and second binding entity types are different from one another.

70. A method according to any one of aspects 58 to 68, wherein the first and second binding entity types are the same.

71. A method according to any one of aspects 58 to 70, further comprising electing the amplification particle to have an index of refraction differing from both the carrier fluid and analyte by more than 0.1.

72. A method according to any one of aspects 58 to 70, further comprising electing the amplification particle to have an index of refraction differing from both the carrier fluid and analyte by more than 0.25.

73. A method according to any one of aspects 58 to 70, further comprising electing the amplification particle to have an index of refraction differing from both the carrier fluid and analyte by more than 0.5.

74. A method according to any one of aspects 58 to 70, further comprising electing the amplification particle to have an index of refraction differing from both the carrier fluid and analyte by more than 1.0.

75. A method according to any one of aspects 58 to 74, wherein the carrier fluid is water.

76. A method according to any one of aspects 58 to 74, wherein the carrier fluid is a bodily fluid.

77. A method according to aspect 76, wherein the bodily fluid is blood.

78. A method according to aspect 76, wherein the bodily fluid is urine.

79. A method according to any one of aspects 58 to 74, wherein the carrier fluid is a buffer solution.

80. A method according to any one of aspects 58 to 74, wherein the carrier fluid is air.

1. A system for performing refractometry, comprising:
a laser configured to output a plurality of optical pulses, each optical pulse having a corresponding selected wavelength and input amplitude;
a detector configured to detect and measure an output signal, the output signal comprising an output amplitude at each of the selected wavelengths;
a primary waveguide, disposed on a substrate, the primary waveguide having an injection end optically coupled to the laser and an output end optically coupled to the detector, the primary waveguide having an interior surface configured to exhibit total internal reflection for each of the optical pulses and an exterior surface;
a secondary waveguide disposed on the substrate, the secondary waveguide comprising an interior, closed-loop optical path exhibiting total internal reflection at the wavelengths of each of the optical pulses, and an exterior surface having bound thereto a plurality of instances of a first binding entity type for an analyte, the first binding entity type configured to bind to the analyte when exposed to the analyte, wherein a portion of the exterior surface of the secondary waveguide is adjacent to the exterior surface of the primary waveguide; and
a solution comprising a carrier fluid and an amplification complex, the amplification complex comprising a secondary particle bound to a second binding entity type for the analyte, the second binding entity type selected to bind to the analyte when exposed to the analyte, wherein the secondary particle is selected to have an index of refraction different from both the carrier fluid and the analyte.

2. A system according to claim 1, wherein the analyte is a bacterial antigen, a viral antigen, or a protein.

3. A system according to claim 1, wherein the secondary particle comprises a metal.

4. A system according to claim 1, wherein the secondary particle comprises silver or gold.

5. A system according to claim 1, wherein the secondary particle comprises a silicate.

6. A system according to claim 1, wherein the first and second binding entity types are different from one another.

7. A system according to claim 1, wherein the first and second binding entity types are the same.

8. A system according to claim 1, wherein the primary and secondary waveguides each have an interior surface and an exterior surface, and wherein the interior surfaces each exhibit total internal reflection at the selected wavelengths.

9. A system according to claim 8, wherein the minimum distance between the primary and secondary waveguides is selected to maximize optical coupling between the first and second waveguides.

10. A system according to claim 1, wherein the secondary particle is selected to have an index of refraction differing from both the carrier fluid and the analyte by more than 0.1.

11. A method of detecting an analyte using an optical ring resonator comprising a primary waveguide and a secondary waveguide, the secondary waveguide comprising a closed-loop optical path disposed adjacent to the primary waveguide, the method comprising:
functionalizing an amplification particle by causing a first binding entity type to bind to the amplification particle, the first binding entity type selected to bind to both the amplification particle and the analyte;
exposing the functionalized amplification particle to a carrier fluid containing, or suspected or containing, the analyte, such that the analyte, if present, binds to the functionalized amplification particle;
functionalizing an exterior surface of the secondary waveguide by causing a second binding entity type to bind to the waveguide, the second binding entity type selected to bind to both the waveguide and the analyte;
exposing the functionalized waveguide to the carrier fluid containing, or suspected of containing, the analyte, such that the analyte, if present, binds to the second binding entity type; and
performing refractometry, using the primary and secondary waveguides, to detect the amplification particle.

12. A method according to claim 11, wherein performing refractometry to detect the amplification particle comprises:
injecting a series of laser optical pulses into a first end of the primary waveguide, each optical pulse having a corresponding selected wavelength and amplitude;
measuring at a detector optically coupled to a second end of the primary waveguide, the output amplitude of each of the pulses to determine a transmission value at each of the selected wavelengths; and
comparing the transmission values at each of the selected wavelengths to corresponding transmission values obtained prior to exposing the functionalized waveguide to the carrier fluid.

13. A method according to claim 11, wherein the analyte is a bacterial antigen, a viral antigen, or a protein.

14. A method according to claim 11, wherein the amplification particle comprises a metal.

15. A method according to claim 11, wherein the amplification particle comprises silver or gold.

16. A method according to claim 11, wherein the amplification particle comprises a silicate.

17. A method according to claim 11, wherein the first and second binding entity types are different from one another.

18. A method according to claim 11, wherein the primary and secondary waveguides each have an interior surface and an exterior surface, and where in the interior surfaces each exhibit total internal reflection at the selected wavelengths.

19. A method according to claim 11, further comprising electing the amplification particle to have an index of refraction differing from both the carrier fluid and analyte by more than 0.1.

20. A method of detecting an analyte using a photonic resonator structure to perform refractometry, the method comprising:
functionalizing an amplification particle by causing a first binding entity type to bind to the amplification particle, the first binding entity type selected to bind to both the amplification particle and the analyte;
exposing the functionalized amplification particle to a carrier fluid containing, or suspected or containing, the analyte, such that the analyte, if present, binds to the functionalized amplification particle;
functionalizing an exterior surface of the photonic resonator structure by causing a second binding entity type to bind to the photonic resonator structure, the second binding entity type selected to bind to both the photonic resonator structure and the analyte;
exposing the functionalized photonic resonator structure to the carrier fluid containing, or suspected of containing, the analyte, such that the analyte, if present, binds to the second binding entity type; and
performing refractometry, using the photonic resonator structure, to detect the amplification particle.
